(19) United States
(12) Patent Application Publication
(10) Pub. No.: US 2009/0276027 A1
(43) Pub. Date: Nov. 5, 2009

(54) Stent Graft Delivery System and Method of Use

(75) Inventor: Brian Glynn, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vasscular, Inc., Santa Rosa, CA (US)

(21) Appl. No.: 12/052,989

(22) Filed: May 1, 2008

(51) Int. Cl. A61F 2/06; A61B 19/00

(52) U.S. Cl. 623/1.11; 623/1.13; 128/898

(57) ABSTRACT

A stent graft delivery system and method of use including a delivery system for a stent graft including a nosecone assembly having a nosecone and a nosecone shaft; a spindle assembly defining a spindle assembly lumen through which the nosecone shaft can slide, the spindle assembly having a spindle fitting and a spindle shaft; and a stent capture assembly defining a stent capture assembly lumen through which the spindle shaft can slide, the stent capture assembly having a stent capture fitting and a stent capture shaft. The spindle fitting is slidably mateable with the stent capture fitting to retain an end of the stent graft at a delivery diameter. A stent graft delivery system with an end stent capture configuration providing both a primary and a secondary deployment procedure facilitated by the threaded connection between a bare stent crown spindle fitting and a system nosecone.

### TECHNICAL FIELD

The technical field of this disclosure is medical implantation devices, particularly, a stent graft delivery system and method of use.

### BACKGROUND OF THE INVENTION

Wide ranges of medical treatments have been developed using endoluminal prostheses, which are medical devices adapted for temporary or permanent implantation within a body lumen, such as naturally occurring or artificially made lumens. Examples of lumens in which endoluminal prostheses may be implanted include lumens such as those located within coronary, mesentery, peripheral, or cerebral vasculature; arteries; gastrointestinal tract; biliary tract; urethra; trachea; hepatic shunts; and fallopian tubes. Various types of endoluminal prostheses have also been developed with a particular structure to modify the mechanics of the targeted vessel wall.

A number of vascular devices have been developed for replacing, supplementing, or excluding portions of blood vessels. These vascular devices include endoluminal vascular prostheses and stent grafts. Aneurysm exclusion devices, such are used to exclude vascular aneurysms and provide a prosthetic lumen for the flow of blood. Vascular aneurysms (abnormal dilation of a blood vessel) are usually the result of disease or a genetic predisposition, which can weaken the arterial wall and allow it to expand. Aneurysms can occur in any blood vessel, but most occur in the aorta and peripheral arteries, with the majority of aneurysms occurring in the abdominal aorta or the aortic arch. An AAA (abdominal aortic aneurysm) typically begins below the renal arteries and extends into one or both of the iliac arteries. A TAA (thoracic aortic aneurysm) typically occurs in the ascending or descending aorta.

Aneurysms, especially abdominal aortic aneurysms, were historically treated with open surgery procedures where the diseased vessel segment is bypassed and repaired with an artificial vascular graft. While open surgery was and is an effective surgical technique in light of the high risk associated with a fatal abdominal aortic aneurysm rupture, the open surgical technique suffers from a number of disadvantages. It is complex, requires a long hospital stay, requires a long recovery time, and has a high mortality rate. Less invasive devices and techniques have been developed to avoid these disadvantages. Tubular endoluminal prostheses that provide a tubular graft for blood flow while excluding blood flow to the aneurysm site are introduced into the blood vessel using a catheter in a less or minimally invasive technique. The tubular endoluminal prosthesis is introduced in a small diameter compressed configuration and expanded at the aneurysm. Often referred to as stent grafts, these tubular endoluminal prostheses are used to secure tubular graft material held open in a sealing engagement with the vessel wall by one or more stents as a support structure.

Stent grafts for use in aortic aneurysms typically include a support structure supporting woven or interlocked graft material. Examples of woven graft materials are woven polymer materials, e.g., Dacron, or polytetrafluoroethylene (PTFE). Interlocked graft materials include knit, stretch, and velour materials. The graft material is secured to the inner or outer diameter of the support structure, which supports the graft material and/or holds it in place against a vessel wall. The stent graft is secured to a vessel wall above and below the aneurysm. An open crown without the graft material can be located above the aneurysm to provide a radial force to engage the vessel wall and seal the stent graft to the vessel wall.

One concern in the deployment of stent grafts is to precisely place the stent graft in the vessel, especially in curving portions of the vasculature such as the aortic arch. When the stent graft is allowed to expand in such curves without constraining the proximal end of the stent graft, it may tilt unpredictably to an undesired position. Such tilting can reduce the effectiveness of the seal and contribute to inaccurate placement. A current practice to minimize these drawbacks is to pull the partially deployed stent graft into better axial alignment. This movement may damage the vessel and some misalignment may remain.

One approach to the alignment problem as described in US Patent Application Publication No. 2006/0276872 to Arbefuielle, et al., has been to restrain the end bare stent [30] of the stent graft while the stent graft expands, then to release the end bare stent [30]. A nose cone [632] fixed to a distal apex head [636] and a guidewire lumen (containing shaft or member) [620] engages the ends of the end bare stent [30] which is trapped by a retractable apex body [638]. To release the bare stent [30] once the stent graft has been expanded, the apex body [638] is pulled back using an apex release lumen (containing member)[640] to which the apex body [638] is connected.

To release the end bare stent [30], the guidewire lumen (containing member) [620] must bear a compressive load opposing the tensile load applied to apex release lumen (containing member) [640] to retract the retractable apex body [638] as the end bare stent [30] is released. The apex release lumen (containing member) [640] connected to the apex body [638] is pulled backward while the guidewire lumen (containing member) [620] connected to the nose cone and the distal apex head [636] is held stationary to oppose the motion of the apex release lumen (containing member [640]. Not holding the guidewire lumen (containing member) [620] stationary can affect the placement of the stent graft. If the nose cone is inadvertently advanced in the vessel, axial misplacement of the stent graft will result and possibly undesired contact with sensitive structure such as the aortic valve.

In the steps of stent graft deployment prior to undertaking release of the end bare stent [30], a large portion of the proximal portion of the stent graft has already been deployed and is in contact with the adjacent vessel wall. Therefore the release of the end bare stent must be assured to be able to release the stent graft from the delivery system and remove the delivery system from the patient. A breakage or disconnection from the delivery handle of either the guidewire lumen (containing member) [620] or the apex release lumen (containing member) [640] will prevent deployment and require that an open surgical repair to remove the delivery system and partially implanted stent graft be undertaken immediately.

It would be desirable to overcome the above disadvantages.

### SUMMARY OF THE INVENTION

One aspect according to the present invention provides a delivery system for a stent graft including a nosecone assembly having a nosecone and a nosecone shaft; a spindle assembly defining a spindle assembly shaft through which the nosecone shaft can slide, the spindle assembly having a spindle fitting and a spindle shaft; and a stent capture assembly defining a stent capture assembly shaft through which the spindle shaft can slide, the stent capture assembly having a stent capture fitting and a stent capture shaft. The spindle fitting is slidably mateable with the stent capture fitting to retain one end of the stent graft at a delivery diameter.

Another aspect according to the present invention provides a method of delivering a stent graft to a deployment site in a vessel including providing a stent graft delivery system; loading a stent graft on the stent graft delivery system with one end of the stent graft over the spindle fitting, the stent capture fitting over the one end of the stent graft, and the stent graft compressed to a delivery diameter; advancing the stent graft delivery system through the vessel to align the spindle fitting with the deployment site; expanding the stent graft while maintaining the one end of the stent graft at the delivery diameter; and pulling the capture fitting shaft against the spindle shaft to retract the stent capture fitting and release the one end of the stent graft. The stent graft delivery system includes a nosecone assembly having a nosecone and a nosecone shaft; a spindle assembly having a spindle fitting and a spindle shaft, the spindle assembly defining a spindle assembly lumen; and a stent capture assembly having a stent capture fitting and a stent capture shaft, the stent capture assembly defining a stent capture assembly lumen. The nosecone shaft is slidably disposed in the spindle assembly lumen and the spindle shaft is slidably disposed in the stent capture assembly lumen.

Another aspect according to the present invention provides a delivery system for a stent graft at a deployment site including means for releasably retaining one end of the stent graft at a delivery diameter; means for advancing the retaining means to the deployment site; and means for retracting the retaining means to release the one end of the stent graft from the delivery diameter.

The foregoing and other features and advantages will become further apparent from the following detailed description, read in conjunction with the accompanying drawings. The detailed description and drawings are merely illustrative.

### BRIEF DESCRIPTION OF THE DRAWINGS

FIG. 1 is a side view of a stent graft;

FIG. 2 is a perspective view of a portion of a nosecone assembly;

FIGS. 3A & 3B are a side and end view, respectively, of a portion of a spindle assembly;

FIG. 3C is a side view of another embodiment of a spindle fitting;

FIGS. 4A & 4B are a side and end view, respectively, of a portion of a stent capture assembly;

FIG. 4C is a side view of another embodiment of a stent capture fitting arm;

FIGS. 5A & 5B are a side view and perspective view, respectively, of a portion of a stent graft delivery system;

FIGS. 6A-6C are detailed perspective views of a portion of a stent graft delivery system;

FIG. 7 is a flowchart of a method of delivering a stent graft to a deployment site in a vessel;

FIG. 8 is a schematicized side view of a partially deployed stent graft,

FIG. 9A and 9B are a partial and tight close-up plan views of another embodiment of a stent graft delivery system with a portion of the bare stent of the stent graft cut away for ease of understanding, where the crowns of the bare stent are captured by a stent graft capture fitting,

FIGS. 10A, 10B, and 10C are a cross sectional views of the delivery system of FIG. 9A and 9B showing progressive steps of stent graft deployment in a primary release mode, and

FIGS. 11A, 11B, 11C, and 11D are a cross sectional views of the delivery system of FIG. 9A and 9B showing progressive steps of stent graft deployment in a secondary release mode.

### DETAILED DESCRIPTION

Embodiments according to the invention will now be described by reference to the figures wherein like numbers refer to like structures. The terms “distal” and “proximal” for the delivery system are used herein with reference to the treating clinician during the use of the stent graft delivery system: “distal” indicates a portion of the stent graft delivery system distant from, or a direction away from the clinician and “proximal” indicates a portion of the stent graft delivery system near to, or a direction towards the clinician. The terms “distal” and “proximal” for the stent graft are used herein with reference to the direction of blood flow from the patient's heart to and through the stent graft device: proximal” indicates a portion of the stent graft nearest the heart according to the blood flow path from the heart to the device, “distal” indicates a portion of the stent graft distant from heart according to blood flow path, or the end opposite the proximal end. In the example provided here the proximal end of the stent graft during delivery corresponds with the distal end of the stent graft delivery system. As defined herein, the deployment site is the axial position in a vessel at which the proximal end of a stent graft is to be located when the stent graft is deployed.

Embodiments according to the invention disclose stent graft delivery devices and methods of use. While these devices and methods are described below in terms of being used to treat abdominal aortic aneurysms and thoracic aortic aneurysms, those skilled in the art will appreciate that the devices could be used to deliver other devices in other vessels as well. Stent graft delivery devices described include stent graft delivery systems for delivering a stent graft to a deployment site in a vessel, with the systems including a spindle fitting and stent capture fitting axially slidable relative to a nosecone shaft and releasably retaining the proximal (in these examples) end of the stent graft at a delivery diameter.

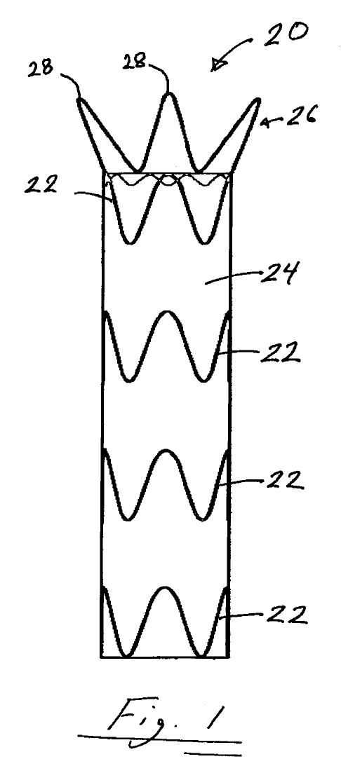

FIG. 1 is a side view of a stent graft for use with a stent graft delivery system. The stent graft 20, illustrated in the deployed state, includes stent 22s and graft material 24 supported by the stents 22. In this example, the stent graft 20 further includes a bare stent (spring) 26 with a number of bare stent crowns 28. The bare stent 26 extends beyond the graft material 24 to provide a radial force which engages the vessel wall and seals the stent graft 20 at the vessel wall. One apex of each stent crown 28 is at the distal end of the stent graft 20. In another embodiment, the bare stent can be omitted. The stent graft 20 is delivered to the deployment site at a delivery diameter and expanded at the deployment site to a deployed diameter.

A stent graft can be described as any suitable device for mechanically keeping a tubular graft open and in sealing contact with healthy surrounding tissue after being implanted at the deployment site, such as a deployment site in the abdominal aorta, thoracic aorta, or other vessel. Such mechanical endoprosthetic devices are typically inserted into the target vessel, positioned across the lesion, and then expanded to bypass the weakened wall of the vessel, thereby preventing rupture of the aneurysm. The stent graft is in contact with the healthy tissue after implantation of the stent graft. The stent graft generally extends across the aneurysm in a vessel to divert flow through the stent graft and relieve the pressure normally applied to the weak aneurysmal wall.

The size and configuration of the stents 22 depend upon the size and configuration of the vessel to be treated. Individual stents 22 can be connected to each other by articulated or rigid joints or can be attached only to the graft material 24. The minimum length of the stent graft 20 to be used is matched (slightly oversized) to the size of the aneurysm across which the stent graft 20 will be implanted.

The stents 22 and the graft material 24 can be any stents and the graft material typically used for stent grafts. The stents 22 can be self-expanding or balloon expandable, and can be a single unit along the whole length of the stent graft or a series of individual stents as illustrated in FIG. 1. The stents 22 can be made of can be made of spring steel, stainless steel, titanium, nickel titanium alloys (Nitinol), a polymer or copolymer, a combination of these materials, or other suitable materials. The graft material 24 can be any woven or interlocked graft material suitable for stent grafts, such as woven polymer materials, e.g., Dacron polyester, or polytetrafluoroethylene (PTFE), or interlocked graft materials including knit, stretch, and velour materials. In some embodiments, the graft material 24 includes components made of collagen, albumin, an absorbable polymer, or biocompatible fiber. Alternatively, the graft material 24 is constructed from one or more suitable metallic, plastic, or non-biodegradable materials.

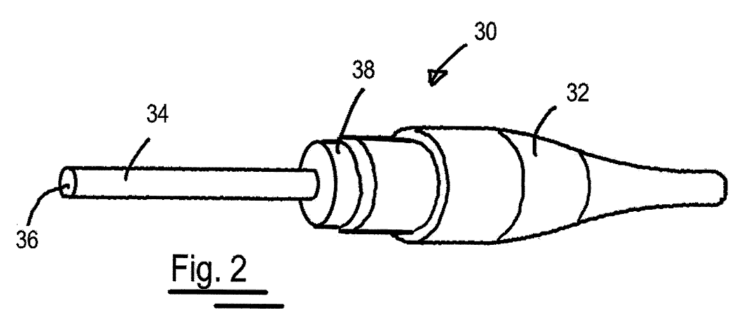

FIGS. 2-4 illustrate the parts of a stent graft delivery system. The stent graft delivery system includes a nosecone assembly, a spindle assembly, and a stent capture assembly. The nosecone assembly has a nosecone and a nosecone shaft, with a nosecone assembly lumen therethrough through which a guidewire can slide. The spindle assembly has a spindle fitting and a spindle shaft, with a spindle assembly lumen therethrough through which the nosecone shaft can slide. The stent capture assembly has a stent capture fitting and a stent capture shaft, and defines a stent capture assembly lumen through which the spindle shaft can slide. The spindle fitting and the stent capture fitting are slidably mateable and releasably retain proximal end of the stent graft at a (compressed) delivery diameter. The spindle fitting, the stent capture fitting, and the nosecone can each move independently and relative to each other, although when moved toward each other and into contact with the adjacent piece, the pieces in contact will move as one. When the spindle fitting engages the stent graft bare spring, the spindle fitting motion is limited by the travel limits imposed by the bare stent, the nosecone and capture fittings. The stent capture fitting can be retracted from the spindle fitting to release the end of the stent graft or the nosecone assembly can be disengaged from the spindle assembly to release the end of the stent graft.

FIG. 2 is a perspective view of a portion of a nosecone assembly. The nosecone assembly 30 includes a nosecone 32 and a nosecone shaft 34, and guides the spindle assembly and stent capture assembly through the vasculature. The nosecone 32 can be generally tapering from the distal to the proximal end to facilitate passage through a vessel. The nosecone shaft 34, which guides the spindle fitting and stent capture fitting to the deployment site, is long enough the reach through the vasculature from the stent graft deployment site in the vessel to the clinician. The proximal end of the nosecone shaft 34 can be attached to a handle (not shown) for manipulation by the clinician during stent graft delivery. In one embodiment, the nosecone assembly 30 defines a guidewire lumen 36 along its length through which a guidewire can slide to guide the delivery system to the deployment site. In another embodiment, the nosecone assembly 30 can include a transition piece 38 adapted to the spindle fitting and the stent capture fitting to assist in retaining one end of the stent graft and facilitate passage through the vasculature. The transition piece 38 can include one or more steps in diameter. The transition piece 38 can include an arm transition segment 37, so that the arms of the stent capture fitting (not shown) can fit around the arm transition segment 37. The diameter of the arm transition segment 37 is sized to receive the stent capture fitting arms and to be smaller than the largest diameter of the nosecone 32 so that the stent capture fitting arms are recessed and protected when passing through the vasculature. The transition piece 38 can also include a catheter transition segment 39, so that a catheter (not shown) can fit around the catheter transition segment 39. The diameter of the catheter transition segment 39 can be selected to match the inner diameter of the catheter, so that the catheter and the nosecone 32 form a smooth profile when passing through the vasculature. In another embodiment, the transition piece can be omitted.

Those skilled in the art will appreciate that the nosecone assembly 30 can made of any biocompatible material and can be formed as a single unit and/or assembled from individual parts. The nosecone 32 can be constructed by insert molding the specific geometry of the nosecone 32 over the nosecone shaft 34. The nosecone material can be an elastomeric material of a specific durometer to provide a flexible tip for the stent graft delivery system. Suitable nosecone materials include Pebax, urethane, silicone, other flexible polymers, and the like. The nosecone 32 may also include a radiopaque additive to provide the clinician with a visible tip when using fluoroscopy guidance to deliver the stent graft within the patient.

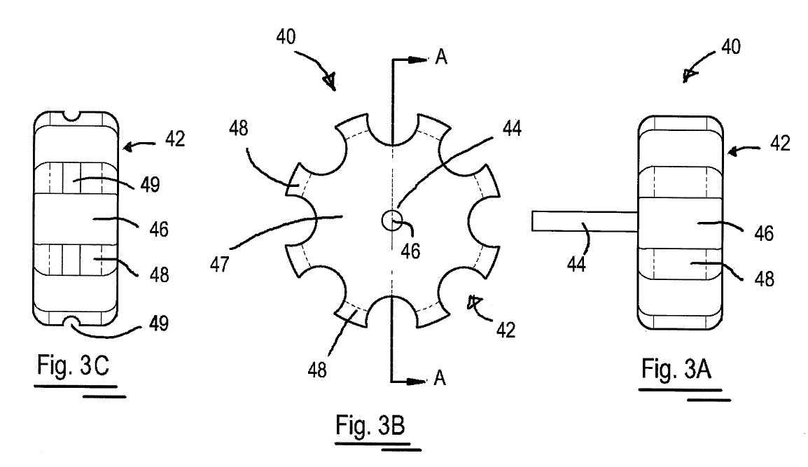

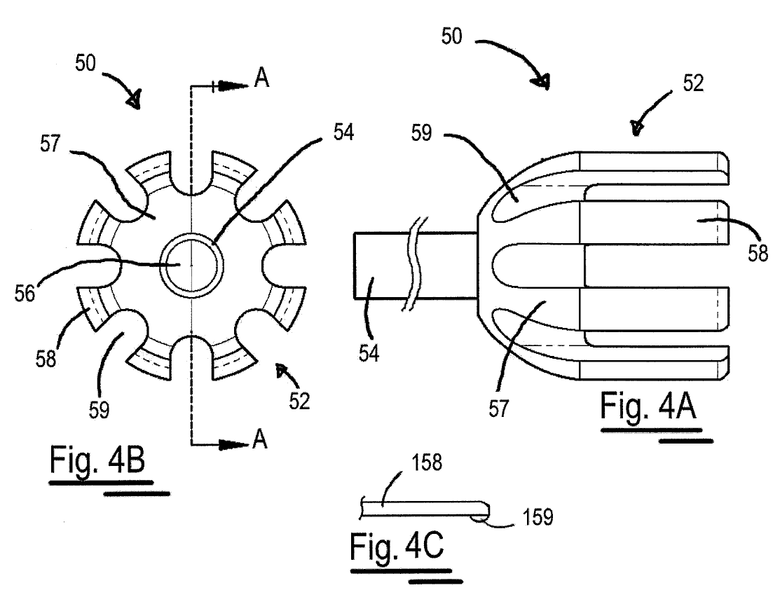

FIGS. 3A & 3B are a side and end view, respectively, of a portion of a spindle assembly. The spindle assembly 40 includes a spindle fitting 42 and a spindle shaft 44. The spindle assembly 40 defines a spindle assembly lumen 46 along its length through which the nosecone shaft (not shown) can slide. The diameter of the spindle assembly lumen 46 is large enough that the nosecone shaft (not shown) can slide within the spindle assembly lumen 46. The spindle shaft 44 advances the spindle fitting 42 over the nosecone shaft to the deployment site. The spindle shaft 44 is long enough the reach through the vasculature from the stent graft deployment site in the vessel to the clinician. The proximal end of the spindle shaft 44 can be attached to a handle (not shown) for manipulation by the clinician during stent graft delivery. Those skilled in the art will appreciate that the spindle assembly 40 can made of any biocompatible material and can be formed as a single unit and/or assembled from individual parts. The spindle shaft can be constructed of a rigid plastic such as PEEK polyetheretherketone, polyimide, nylon, or the like. The spindle shaft can alternatively be constructed of a flexible metal tube such as nitinol, stainless steel, or the like.

The spindle fitting 42, in cooperation with the stent capture fitting (not shown), retains one end of the stent graft during stent graft delivery. In the illustrated embodiment, the spindle fitting 42 includes a spindle body 47 and a number of spindle pins 48 disposed around the circumference of the spindle body 47. A spindle groove 49 is formed between each pair of adjacent spindle pins 48. A single stent crown (not shown) wraps around each spindle pin 48 and is held in place by a stent capture fitting arm (not shown) during stent graft delivery. When the stent capture fitting is retracted, the stent crowns are freed from the spindle pins 48 and the stent crown expands into position in the vessel. The spindle fitting 42 can be made of any rigid and/or compliant biocompatible material and can be formed as a single unit and/or assembled from individual parts. The spindle fitting can be fabricated from a variety of materials. This may include rigid plastic materials such as PEEK polyetheretherketone, polycarbonate, or the like, and may also include metals such as stainless steel. In one embodiment, a hard plastic is desirable for the spindle fitting to avoid damage to the stent surface, which is in contact with the spindle fitting. The spindle fitting can be fastened to the spindle shaft by bonding the two with adhesive or threading the two components together. The spindle fitting may alternatively be insert molded directly on the spindle shaft.

FIG. 3C is a side view of another embodiment of a spindle fitting. In this embodiment, each of the spindle pins 148 on the spindle body 147 includes a spindle slot 149 along the spindle pin circumference of the spindle pins 148. The spindle pin circumference is defined by the ends of the spindle pins 148 away from the spindle body 147. The distal end of each stent crown, i.e., the apex of each bare stent, rests in one of the spindle slots 149 and the stent capture fitting arm retains the stent crown in the spindle slot 149. The stent capture fitting positively retains the stent crown in the spindle slot 149 until the stent capture fitting is retracted.

In another embodiment, the spindle fitting can be a compliant disc of a uniform circumference and omitting the spindle pins. The stent crowns can be pressed into the compliant disc by the stent capture fitting arm to hold the stent crown compressed during stent graft delivery. When the stent graft does not include a bare stent, the stent capture fitting arms can press the distal end of the stent graft (both the stent and the graft material) into the compliant disc. The graft material can be stretchable or loose on the stents to allow the graft material to extend around the stent capture fitting arms when the stent capture fitting arm holds the distal end of the stent compressed. The compliant disc can be made of a low durometer polymer such as silicone. In yet another embodiment, the spindle fitting can be molded to include additional features that match the specific shape of the compressed stent. In one example, the spindle pins may have a tapered profile that matches the curvature of the compressed stent crown.

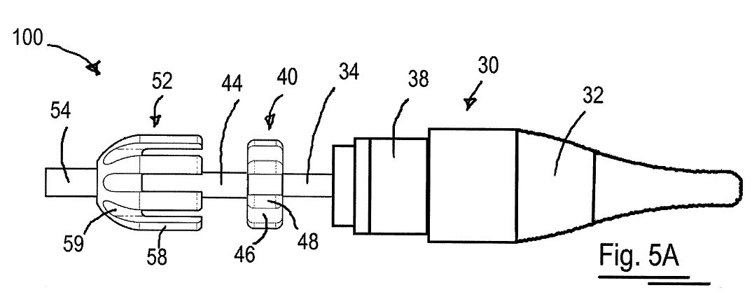

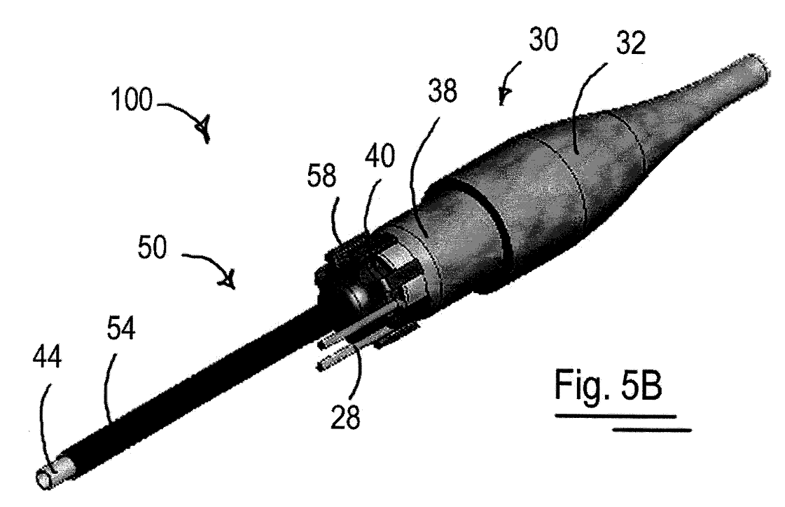

FIGS. 4A & 4B are a side and end view, respectively, of a portion of a stent capture assembly. The stent capture assembly 50 includes a stent capture fitting 52 and a stent capture shaft 54. The stent capture assembly 50 defines a stent capture assembly lumen 56 along its length through which the spindle shaft (not shown) can slide. The diameter of the stent capture assembly lumen 56 is large enough that the spindle shaft (not shown) can slide within the stent capture assembly lumen 56. The stent capture shaft 54 advances the stent capture fitting 52 to the deployment site and retracts the stent capture fitting 52 to release the end of the stent graft from the delivery diameter. The stent capture shaft 54 is long enough the reach through the vasculature from the stent graft deployment site in the vessel to the clinician. The proximal end of the stent capture shaft 54 can be attached to a handle (not shown) for manipulation by the clinician during stent graft delivery. Those skilled in the art will appreciate that the stent capture assembly 50 can made of any biocompatible material and can be formed as a single unit and/or assembled from individual parts. The stent capture shaft may be constructed of a rigid plastic, such as PEEK polyetheretherketone, polyimide, nylon, or the like. The stent capture shaft can alternatively be constructed of a flexible metal tube such as nitinol, stainless steel, or the like.

The stent capture fitting 52 in cooperation with the spindle fitting (not shown), retains one end of the stent graft during stent graft delivery. In the illustrated embodiment, the stent capture fitting 52 includes a stent capture body 57 having a number of stent capture fitting arms 58, disposed around the circumference of the stent capture body 57. The stent capture body 57 defines a number of stent capture grooves 59 between each of the stent capture fitting arms 58 to receive the bare stent crowns. The stent capture fitting arms 58 can be substantially parallel to the central axis of the stent capture fitting 52, i.e., the axis along the stent capture shaft 54. In other embodiments, the stent capture fitting arms 58 can curve toward or away from the axis of the stent capture fitting 52 as desired for a particular purpose. When the stent capture fitting 52 is retracted, the stent capture fitting arms 58 release the bare stent crowns, and the bare stent crowns expand into position in the vessel. The stent capture fitting 52 can be made of any rigid and/or compliant biocompatible material and can be formed as a single unit and/or assembled from individual parts. The stent capture fitting may be fabricated from a variety of materials. This may include rigid plastic materials such as PEEK polyetheretherketone, polycarbonate, or the like, and may also include metals such as stainless steel. In one embodiment, a hard plastic or highly polished metal is desirable for the stent capture fitting to avoid damage to the stent surface which is in contact with the stent capture fitting. The stent capture fitting can be fastened to the stent capture shaft by bonding the two with adhesive or threading the two components together. The stent capture fitting may alternatively be insert molded directly on the stent capture shaft.

FIG. 4C is a side view of another embodiment of a stent capture fitting arm. The distal end of each of the stent capture fitting arms 158 can include a protrusion 159 projecting inwardly toward the central axis of the stent capture fitting. The protrusions 159 can be large enough to positively retain the distal end of the bare stent crown on the stent capture fitting arm, but small enough to allow the stent capture fitting arm 158 to be retracted over the distal end of the bare stent crown.

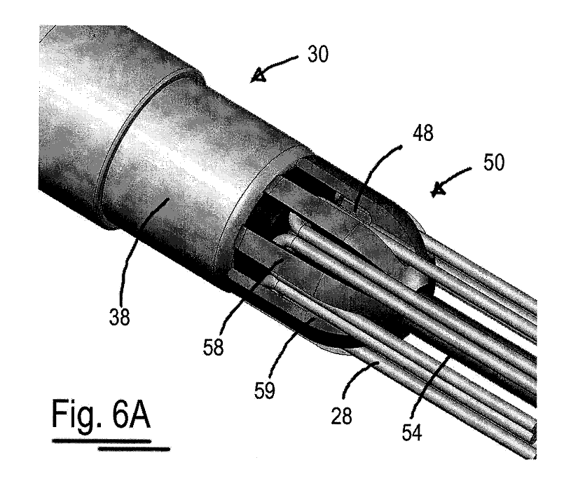

FIGS. 5A & 5B are a side view and a partial perspective view, respectively, of a portion of a stent graft delivery system. FIG. 5A illustrates the stent graft delivery system components slid apart in preparation for loading a stent graft and FIG. 5B illustrates the components slid together. FIG. 5B shows one bare stent crown 28 in the loaded position and omits the remainder of the stent graft for clarity of illustration. The stent graft delivery system 100 includes nosecone assembly 30, spindle assembly 40, and stent capture assembly 50. The spindle assembly 40 is slidably disposed over the nosecone shaft 34 of the nosecone assembly 30 and the stent capture assembly 50 is slidably disposed over the spindle shaft 44. In this example, the stent capture fitting arms 58 of the stent capture fitting 52 extend onto the transition piece 38 of the nosecone assembly 30. Those skilled in the art will appreciate that the stent capture fitting arms 58 only need extend far enough to secure the stent crowns 28 on the spindle assembly 40 and need not extend onto the transition piece 38. The proximal ends of the nosecone assembly 30, spindle assembly 40, and stent capture assembly 50 can terminate in a handle which allows the clinician to slide each of the shafts independently of each other and to advance the shafts through the vasculature as a group. The stent graft delivery system 100 can also include a graft cover (or sheath) (not shown) slidable over the stent capture assembly 50 and the stent graft when the proximal end of the stent graft is retained between the spindle fitting 42 and the stent capture fitting 52. The graft cover can hold the stent graft at a compressed delivery diameter until deployed.

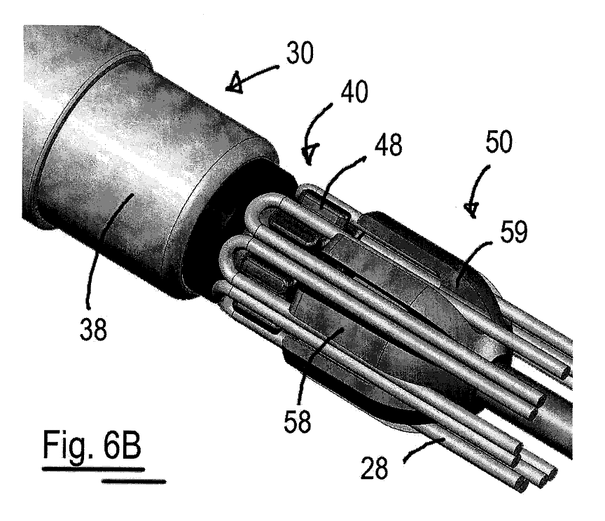

FIGS. 6A-6C are detailed perspective views of a portion of a stent graft delivery system. FIG. 6A illustrates the end of the stent graft retained between the spindle fitting and the stent capture fitting; FIG. 6B illustrates the stent capture fitting retracted from the spindle fitting ; and FIG. 6C illustrates the nosecone pushed forward away from the spindle fitting and the stent capture fitting.

Referring to FIG. 6A, the stent graft is loaded in the stent graft delivery system, with the bare stent crowns 28 about the spindle pins 48 of the spindle fitting. The stent capture fitting arms 58 extend through the grooves 59 of the stent capture fitting 52. In this example, the distal ends of the stent capture fitting arms 58 extend onto the of the nosecone assembly 30. The apex of each bare stent crown 28 is trapped by the stent capture fitting arm 58, the spindle pin 48, and the arm transition segment 37.

Referring to FIG. 6B, the stent capture fitting 52 is illustrated in a retracted position, so that the stent capture fitting arms 58 are withdrawn from the spindle pins 48 of the spindle assembly 40 and no longer positioned to trap the bare stent crowns 28. The bare stent crowns 28 are shown at the compressed delivery diameter for clarity of illustration, while actually the stent crowns 28 when no longer trapped would have self expanded to the deployment diameter when the stent capture fitting 52 was retracted and the stent graft is free of the graft cover.

Referring to FIG. 6C, the nose cone 32 is pushed forward away from both the spindle fitting and the stent capture fitting 52. The nosecone assembly, spindle assembly, and stent capture assembly are slidable independently of each other, so the position of the nose cone 32 can be adjusted relative to the deployment site without moving spindle fitting and the stent capture fitting 52. This allows deployment of the bare stent crowns by providing force on the nosecone shaft of the nosecone assembly when the stent capture fitting 52 cannot be retracted from the spindle assembly 40. The bare stent crowns 28 exert an outward radial force against the distal ends of the stent capture fitting arms 58 to hold the distal ends of the bare stent crowns 28 at the delivery diameter until the nosecone is pushed forward to release the bare stent crowns.

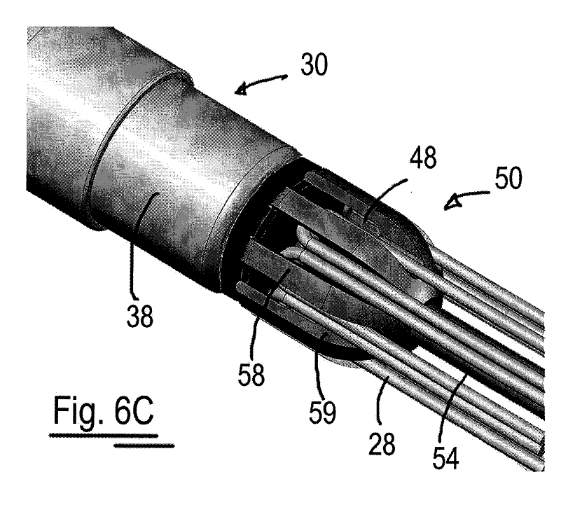

FIG. 7 is a flowchart of a method of delivering a stent graft to a deployment site in a vessel. The deployment site can be located in an abdominal aorta, a thoracic aorta, or any other vessel. The method 200 includes the step of providing a stent graft delivery system (202) including a nosecone assembly having a nosecone and a nosecone shaft; a spindle assembly having a spindle fitting and a spindle shaft the spindle assembly defining a spindle assembly lumen; and a stent capture assembly having a stent capture fitting and a stent capture shaft, the stent capture assembly defining a stent capture assembly lumen. The nosecone shaft is slidably disposed in the spindle assembly lumen and the spindle shaft is slidably disposed in the stent capture assembly lumen. The method 200 further includes the steps of: loading a stent graft on the stent graft delivery system (204) with one end of the stent graft over the spindle fitting, the stent capture fitting over the one end of the stent graft, and the stent graft compressed to a delivery diameter; advancing the stent graft delivery system through the vessel (206) to align the spindle fitting with the deployment site; expanding the stent graft (208) while maintaining the one end of the stent graft at the delivery diameter; and pulling the capture fitting shaft against the spindle shaft (210) to retract the stent capture fitting and release the one end of the stent graft. In one embodiment, the nosecone assembly defines a guidewire lumen, and advancing the stent graft delivery system through the vessel (206) includes advancing a guidewire through the vessel; inserting the guidewire in the guidewire lumen; and advancing the stent graft delivery system over the guidewire.

The stent capture assembly normally can be moved without applying any force to the nosecone assembly, but when the connection between the stent capture fitting and the deployment handle become inoperative (for whatever reason) the nosecone can be moved forward to effect deployment. Advancing the stent graft delivery system through the vessel (206) can include sliding the stent capture assembly and the spindle assembly on the nosecone shaft until the spindle fitting is aligned with the deployment site. In one embodiment, the method 200 can further include sliding the stent capture assembly and the spindle assembly on the nosecone shaft to realign the spindle fitting with the deployment site before pulling the capture fitting shaft relative to the spindle shaft to effect release of the bare stent crowns and the stent graft.

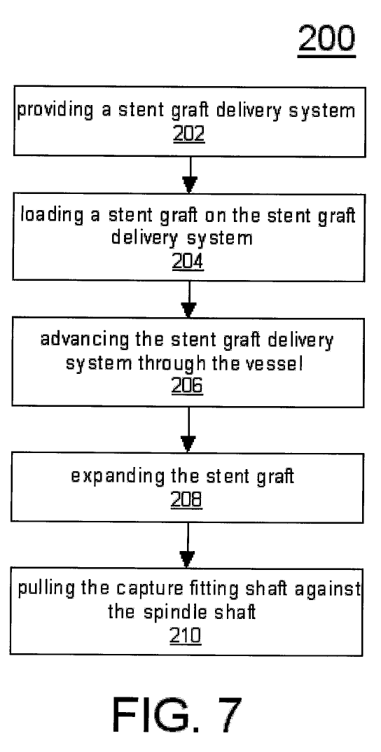

Expanding the stent graft (208) while maintaining the proximal end of the stent graft the delivery diameter can includes retracting a graft cover 90 to release the stent graft, as illustrated in FIG. 8.

FIG. 8 is a side view of a partially deployed stent graft. The graft cover 90 is illustrated being retracted to release the stent graft 20, which is expanding from a compressed delivery diameter to the expanded deployed diameter. The graft cover 90 can releasably maintain the stent graft at the compressed delivery diameter for delivery through the vasculature. The distal end 92 of the stent graft 20 is retained at a delivery diameter by the stent capture fitting 52. After the stent graft 20 is free of the graft cover 90 and the spindle fitting (not shown) is precisely aligned with the deployment site, the stent capture fitting 52 can be retracted to release the distal end 92 of the stent graft.

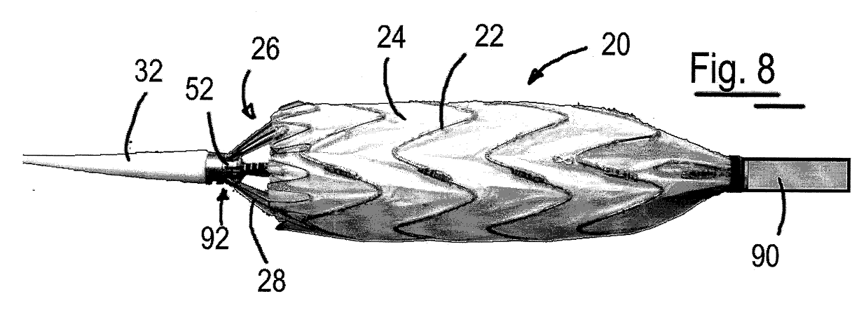

FIG. 9A and 9B show a partial and tight plan views of an embodiment of a stent graft delivery system using only two longitudinally movable pieces, which move relative to one another. While the basic concept of two longitudinally movable pieces has been previously discussed, the details and execution disclosed herein are previously unknown. A nosecone shaft 35 (not seen in FIGS. 9A and 9B) connects to a nosecone 62. A stent capture shaft 55 connected to a stent capture fitting 60 surrounds the nosecone shaft 35, thereby eliminating any stent crown escape gap therebetween, and is configured to slide relative to it as the stent capture fitting 60 engages the nosecone 62 and its spindle fitting 43 (not seen in FIGS. 9A and 9B).

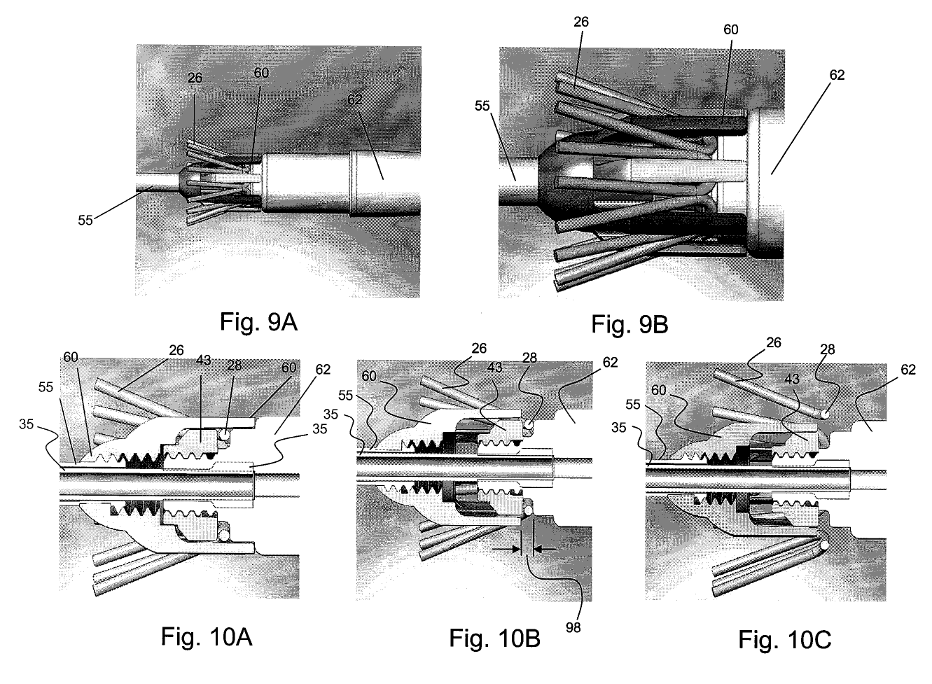

In viewing the cross section of the delivery system shown in FIGs. FIGS. 10A, 10B, and 10C, the bare stent 26 of the stent graft is cut away for clarity and the progressive views show a primary mode of deployment. The nosecone shaft 35 has an integral bulb at it end to hold the nosecone 62. The nosecone 62 is over molded onto the nosecone shaft to form a unitary piece. A lower hub portion of the nosecone 62 has threads formed on its outer surface. A spindle fitting 43, having a similar perimeter configuration to the spindle fitting 42, described in FIGS. 3A, 3B, and 3C, has a central opening having female threads configured to engage the threads on the lower hub of the nosecone 62, such that when the threads of the spindle fitting 43 and the threads of the nosecone 62 are fully engaged, they together with the nosecone shaft 35 move as one unitary piece. The stent capture shaft 55 is at its end is threadably fixed to stent capture fitting 60, and they move as a unitary piece. The stent capture fitting is configured substantially as previously described stent capture fitting 52 in FIGS. 4A, 4B, and 4C. Thus in the primary mode of deployment, the two unitary pieces: the nosecone capture shaft 35, the nosecone 62, and the spindle fitting 43; and the stent capture shaft 55 and the stent capture fitting 60 can move relative to one another longitudinally along the axis of the catheter. When the crown 28 of the bare stent 26 is captured by and between the top of the spindle fitting 43, the bottom and outside of the nosecone 62 across and adjacent to the spindle fitting 43, and the inside of the stent capture fitting arms of the stent capture fitting 60 to hold each of the crowns 28 of the bare stent 26 as the lower portion of the stent graft is deployed causing the struts of the bare stent 26 to pivot around their captured crowns 28 as is pictured in FIG. 10A. In the progression of the primary deployment mode, the lower portion of the stent graft having already been at least partially deployed to contact the adjacent vessel wall and become partially, if not fully, fixed at that particular deployment location in the vessel. The longitudinal position of the captured crowns is therefore substantially fixed within the limits of movement of the bare stent with respect to the main stent graft body portion to which it attaches. Once the stent graft has been partially deployed the crowns 28 can no longer move longitudinally, they can only pivot outward. During primary deployment the stent capture shaft 55 is pulled causing the stent capture fitting 60 to be retracted and open a primary deployment gap 98 (FIG.10B) between the nosecone 62 and the stent capture fitting 60 permitting the crowns 28 of the bare stent 26 to pivot outward to complete deployment as seen in FIG. 1C.

However, when executing the steps of primary deployment FIGS. 10A, 10B, and 10C, it is possible that the connection from a catheter handle (not shown) to the stent capture shaft 55 or that the stent capture shaft 55 itself is broken such that longitudinal force cannot be exerted to retract the stent capture fitting 60 to create the escape (primary deployment procedure) gap 98 shown in FIG. 10B. In the instance when the stent capture fitting 60 is immovable, a one piece nosecone and spindle fitting as has been seen in the art would prevent the crowns 28 of the bare stent form being deployed. Thus requiring, an open surgical intervention to access the site to correct the situation by manual manipulation of the device to complete deployment, or removal of the device and implantation of a standard surgical graft with all the risks and complications associated with an open surgical procedure.

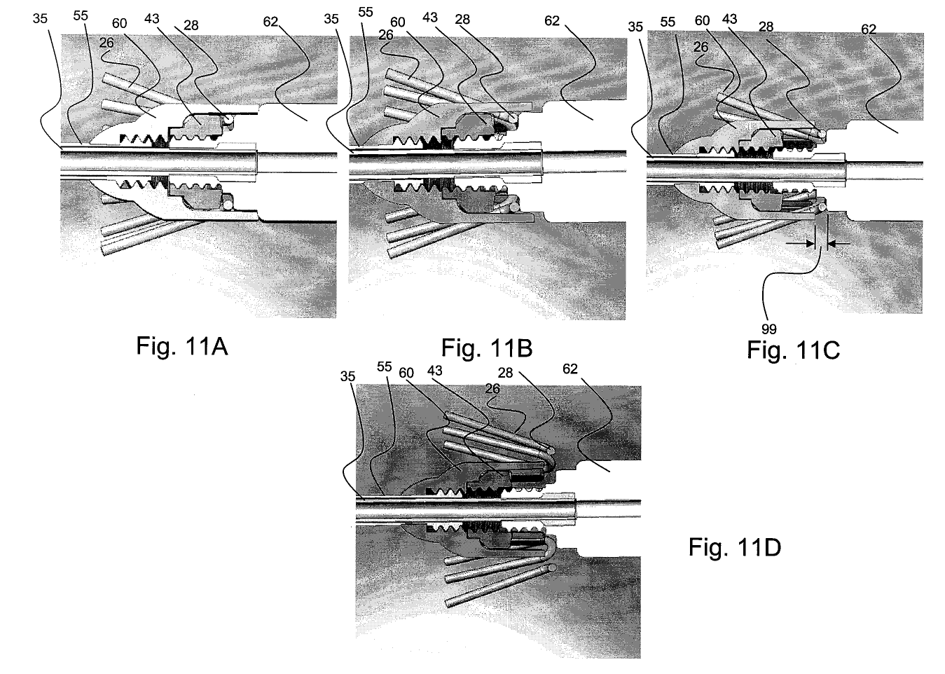

The device described here overcomes the failing of a one piece nosecone device. A secondary deployment procedure shown in FIGS. 11A, 11B, 11C, and 11D shows how the current device overcomes the above described deficiency. FIG. 11A shows the stent crown fully captured similar to that shown and described for FIG. 10A above. Upon the realization that the stent capture shaft 55 cannot be retracted, the operator can initiate the secondary deployment procedure. The partially deployed stent graft in contact with the wall of the vessel acts as both a resistance or a substantial stop to longitudinal movement of the bare spring 26 and also to rotational movement of the bare springs 26. The crowns 28 of the bare spring 26 are positioned around the top of each pin of the spindle fitting 43, which substantially prevents the longitudinal movement of the spindle fitting 43 up or away from the stent graft, while the crowns 28 are captured within the stent capture fitting 60. However the threaded connection between the spindle fitting 43 and the hub of the nosecone 62, previously described, can used to separate the two. With the rotational position of the spindle fitting 43 being set by its engagement with the bare stent which is substantially fixed to the wall of the surrounding vessel, as previously described, a rotational torque can be applied to the nosecone shaft 35 to cause the nosecone 62 to turn and separate from the spindle fitting 43 by the longitudinal action of the threads as the nosecone is turned. This initiation of longitudinal movement of the nosecone 62 away from the spindle fitting 43 is shown in FIG. 11B. Because the bare stent 26 has an unrestrained configuration that is approximately cylindrical, the crowns 28 of the bare stent 26 are urged outward and forward by internal forces which tend to return the bare stent 26 to its unrestrained configuration. As the nosecone 62 continues to be turned with respect to the spindle fitting 43, as shown in FIG. 11C, a secondary deployment procedure gap 99 between the nosecone 62 and the spindle fitting 43 provides a opening allowing the crowns 28 of the bare stent 26 to move forward and escape to provide full release of the crowns 28 of the bare stent 26 as shown in FIG. 11D. The crowns 28 of the bare stent 26 have continued to pivot forward because of the bare stent's internal (spring) forces urging its return to its large diameter unrestrained configuration. While the spindle fitting is now released from the nosecone 62, it is still captured on the nosecone shaft 35 and will be safely removed as the delivery system is now released from the partially deployed stent graft, it having now been fully deployed. Having a primary and a secondary deployment procedure usable with one delivery system to release crowns of a partially deployed stent provides a utility not previously known in the art.

While specific embodiments according to the invention are disclosed herein, various changes and modifications can be made without departing from its spirit and scope.

1. A delivery system for a stent graft comprising:
a nosecone assembly having a nosecone and a nosecone shaft;
a spindle assembly defining a spindle assembly lumen through which the nosecone shaft can slide, the spindle assembly having a spindle fitting and a spindle shaft; and
a stent capture assembly defining a stent capture assembly lumen through which the spindle shaft can slide, the stent capture assembly having a stent capture fitting and a stent capture shaft;
wherein the spindle fitting is slidably mateable with the stent capture fitting to retain one end of the stent graft at a delivery diameter.

2. The delivery system of claim 1 wherein the spindle fitting comprises a spindle body having a circumference, and a plurality of spindle pins disposed around the circumference.

3. The delivery system of claim 2 wherein ends of the plurality of spindle pins away from the spindle body define a spindle pin circumference, and each of the plurality of spindle pins has a spindle slot along the spindle pin circumference.

4. The delivery system of claim 1 wherein the stent capture fitting comprises a stent capture body having a circumference and a plurality of stent capture fitting arms disposed around the circumference substantially parallel to a central axis of the stent capture fitting along the stent capture shaft.

5. The delivery system of claim 4 wherein the stent capture body defines stent capture grooves between adjacent stent capture fitting arms.

6. The delivery system of claim 4 wherein each of the plurality of stent capture fitting arms has a protrusion projecting inwardly toward the central axis.

7. The delivery system of claim 1 further comprising a catheter slidable over the stent capture assembly and the stent graft.

8. A method of delivering a stent graft to a deployment site in a vessel, the method comprising:
providing a stent graft delivery system comprising:
a nosecone assembly having a nosecone and a nosecone shaft;
a spindle assembly having a spindle fitting and a spindle shaft the spindle assembly defining a spindle assembly lumen; and
a stent capture assembly having a stent capture fitting and a stent capture shaft, the stent capture assembly defining a stent capture assembly lumen;
wherein the nosecone shaft is slidably disposed in the spindle assembly lumen and the spindle shaft is slidably disposed in the stent capture assembly lumen;
loading a stent graft on the stent graft delivery system with one end of the stent graft over the spindle fitting, the stent capture fitting over the one end of the stent graft, and the stent graft compressed to a delivery diameter;
advancing the stent graft delivery system through the vessel to align the spindle fitting with the deployment site;
expanding the stent graft while maintaining the one end of the stent graft at the delivery diameter; and
pulling the capture fitting shaft against the spindle shaft to retract the stent capture fitting and release the one end of the stent graft.

9. The method of claim 8 wherein the advancing the stent graft delivery system through the vessel further comprises sliding the stent capture assembly and the spindle assembly on the nosecone shaft until the spindle fitting is aligned with the deployment site.

10. The method of claim 8 further comprising sliding the stent capture assembly and the spindle assembly on the nosecone shaft to realign the spindle fitting with the deployment site before the pulling the capture fitting shaft against the spindle shaft.

11. A delivery system for a stent graft comprising:
a nosecone shaft having a nosecone fixed to an end thereof,
a spindle fitting threadably fixed to and releasable from said nosecone,
a stent capture shaft having a stent capture fitting fixed thereto, the stent capture fitting having arms which surround said spindle fitting and a extend to a position eliminating a gap for stent crown release between the stent capture fitting to said nosecone,
wherein a stent crown is captured between the nosecone, spindle fitting, and stent capture fitting prior to deployment, wherein a primary deployment is achieved by a sliding retraction of the stent capture shaft and stent capture fitting to create a primary deployment gap between said stent capture fitting and said nosecone, wherein a secondary deployment is achieved by rotating said nosecone shaft to provide a threaded progression of the nosecone forward away from the spindle fitting and said stent capture fitting to create a secondary deployment gap between said stent capture fitting and said nosecone.
